(19) United States
(12) Patent Application Publication
(10) Pub. No.: US 2012/0095060 A1
(43) Pub. Date: Apr. 19, 2012

(54) METHODS AND COMPOSITIONS OF MITOGEN-ACTIVATED PROTEIN KINASE (MAPK) PATHWAY INHIBITORS FOR TREATING PULMONARY FIBROSIS

(76) Inventor: William D. HARDIE, Lebanon, OH (US)

(21) Appl. No.: 13/273,586

(22) Filed: Oct. 14, 2011

Related U.S. Application Data
(60) Provisional application No. 61/393,046, filed on Oct. 14, 2010.

(51) Int. Cl. A61K 31/4412; A61P 11/00

(52) U.S. Cl. 514/346

(57) ABSTRACT

Methods and compositions of inhibitors of MAPK kinases 1 and 2 (MEK1 and MEK2, or together, MEK1/2), such as ARRY142886, and their use in inhibiting MEK1/2 kinase activity in mammals for the treatment and/or reversal of the symptoms of pulmonary fibrosis. MEK1/2 inhibitors can selectively inhibit the MAPK/ERK pathway in vivo and prevent TGF-α mediated fibrosis. There are several distinct MAPK pathways that are important in the regulation of cell proliferation, differentiation, development, inflammation, survival, and migration. The Ras-Raf-MEK-ERK pathway (a key component of the MAPK pathway) via inhibition of MEK1/2 is an attractive strategy for therapeutic intervention in idiopathic pulmonary fibrosis, because inhibition of MEK1/2 has the potential to block inappropriate signal transduction leading to pulmonary fibrosis, regardless of the upstream position of the aberration causing it.

### CROSS-REFERENCE TO RELATED APPLICATION

This application claims priority to U.S. Provisional Application No. 61/393,046, filed Oct. 14, 2010, the disclosure of which is incorporated herein by reference in its entirety.

### INTEREST

This invention was made with Government support awarded by the National Institute of Health (NIH), Grant No. HL086598. The Government may have certain rights in this invention.

### FIELD OF THE INVENTION

The present invention relates in general to MAPK pathway inhibitors, and in particular to the methods of using ARRY142886 and related compounds to prevent and treat pulmonary fibrosis.

### BACKGROUND OF THE INVENTION

Pulmonary fibrosis contributes to morbidity and mortality in a number of pediatric and adult lung diseases. Clinical diseases causing pulmonary fibrosis are heterogeneous and include connective tissue disorders, occupational and environmental exposures and interstitial lung diseases (ILD). Also, fibrosis may develop secondary to acute lung injury such as in acute respiratory distress syndrome, from chronic inflammatory diseases such as in cystic fibrosis (CF), or may be of unknown cause as in idiopathic pulmonary fibrosis (IPF). While the pathologic features of pulmonary fibrosis may vary depending on the underlying disease process, they all have a number of common characteristics and processes in common, including mesenchymal cell proliferation, expansion of the extracellular matrix, and remodeling of the lung parenchyma.

Currently there are no proven therapies that prevent or reverse pulmonary fibrosis, emphasizing the need to identify new molecular targets. The molecular pathways and cellular mechanisms leading to pulmonary fibrosis are complex and likely multifactorial. Animal models overexpressing the cytokines TNFα, GM-CSF, IL-11, IL-13 and IL-1β develop varying degrees of pulmonary fibrosis associated with inflammation. In addition, several growth factors regulate matrix deposition and fibroblast proliferation in the lung, including connective tissue growth factor, platelet-derived growth factor (PDGF), basic fibroblast growth factor, insulin-like growth factor and transforming growth factor-β1 (TGF-β1). Transforming growth factor-alpha (TGF-α), along with epidermal growth factor (EGF) and amphiregulin, are ligands for the epidermal growth factor receptor (EGFR).

The epidermal growth factor receptor (EGFR) is a ubiquitous, highly conserved 170-kDa membrane-spanning glycoprotein that is expressed by many cell types in the lung, including lung epithelium, smooth muscle cells, endothelium, and fibroblasts. EGFR is a receptor-linked tyrosine kinase that is activated by extracellular ligands. Ligands for EGFR found in the lung include TGF-α, EGF, amphiregulin, and heparin-binding EGF. Several experimental studies have identified a role for EGFR and its ligands in the pathogenesis of pulmonary fibrosis. TGF-α knockout mice (i.e., mice missing the gene for TGF-α) have significantly reduced lung collagen accumulation compared with wild-type mice following bleomycin injury.

TGF-α activation of EGFR regulates diverse cellular functions, many of which are associated with fibrogenesis, including cell growth, proliferation, differentiation, migration, protection from apoptosis, and transformation. The signaling pathways downstream of EGFR activation which mediate TGF-α induced pulmonary fibrosis are not completely understood. Binding of TGF-α (or EGF) to EGFR causes localized tyrosine kinase activity, leading to autophosphorylation of EGFR tyrosine residues in the cytoplasmic domain. The phosphorylated EGFR tyrosine residues become docking sites for signaling molecules, including MAPK, Src kinases, STAT and the phosphatidylinositol 3-kinase pathway (PI3K), which then activate multiple downstream effector pathways.

Doxycycline (Dox) regulatable transgenic mice that specifically express TGF-α in the lung epithelium show progressive and extensive vascular adventitial, peribronchial, interstitial and pleural fibrosis that is independent of inflammation. Indeed, gene expression profiles observed after expression of TGF-α in these mice lungs are similar to those found in pulmonary fibrotic disease in humans.

While the molecular pathways and cellular mechanisms leading to fibrosis are slowly being elucidated, overall they still remain poorly understood. Current therapy is often ineffective or prohibitive due to side effects, emphasizing the need to identify new therapeutic targets.

In light of the above, there is a need for providing new methods and compositions for treating pulmonary fibrosis. Further, it would be beneficial to determine the role of the EGFR activation in the pathogenesis of pulmonary fibrosis, and to assess the efficacy of therapies designed to inhibit its activity. It would also be beneficial to identify new molecular targets in the MAPK pathways which can be useful in providing new therapies that prevent pulmonary fibrosis. It would also be beneficial to inhibit progression of, and/or reverse, ongoing pulmonary fibrosis caused by expression of TGF-α and increased EGFR signaling.

### SUMMARY OF THE INVENTION

The present invention relates to the discovery that inhibition of the Ras-Raf-MEK-ERK pathway (a key component of the MAPK pathways) via inhibition of MAPK kinases 1 and 2 (MEK1 and MEK2, or together, MEK1/2) is an attractive strategy for therapeutic intervention in idiopathic pulmonary fibrosis. This is because inhibition of MEK1/2 has the potential to block inappropriate signal transduction leading to pulmonary fibrosis, regardless of the upstream position of the aberration causing it.

A first aspect of the invention relates to a method of ameliorating symptoms associated with pulmonary fibrosis in a subject diagnosed with pulmonary fibrosis, comprising the step of administering to the subject a therapeutically effective amount of a MEK1/2 kinase inhibitor, thereby ameliorating the symptoms associated with pulmonary fibrosis in the subject. In one embodiment, the MEK1/2 kinase inhibitor is ARRY142886.

A second aspect of the invention is a method of preventing the progression of established pulmonary fibrosis in a subject diagnosed with pulmonary fibrosis, comprising the step of administering to the subject a therapeutically effective amount of a MEK1/2 kinase inhibitor, thereby preventing the progression of pulmonary fibrosis in the subject. This method can be performed when the pulmonary fibrosis is pronounced, and also when the pulmonary fibrosis is progressing.

A third aspect of the invention is a method of reversing established pulmonary fibrosis in a subject diagnosed with pulmonary fibrosis, comprising the step of administering to the subject a therapeutically effective amount of a composition comprising a MEK1/2 kinase inhibitor, thereby reversing pulmonary fibrosis in the subject.

The method of administering a therapeutically effective amount of a MEK1/2 kinase inhibitor can prevent progressive weight loss, decrease progression of lung fibrosis, and slow progression of TGF-α dependent changes in lung mechanics. In one embodiment, the fibrosis is a non-inflammatory type of fibrosis. In one embodiment, the specific type of fibrosis being prevented and/or treated is idiopathic pulmonary fibrosis.

While the nature and advantages of the present invention will be more fully appreciated from the following drawings and detailed description, showing the contemplated novel construction, combinations and elements as herein described, and more particularly defined by the appended claims, it is understood that changes in the precise embodiments of the present invention are meant to be included within the scope of the claims, except insofar as they may be precluded by the prior art.

### BRIEF DESCRIPTION OF THE DRAWINGS

FIG. 1 is a diagram showing key components of the MAPK/ERK signaling pathways, and inhibitors of those components.

FIG. 2 is a Western blot of lung homogenates (top) showing that CCSP/TGF-α mice administered the MEK1/2 kinase inhibitor ARRY142886 (“ARRY”) demonstrate reduced phosphorylation of ERK1/2 compared with vehicle-treated CCSP/TGF-α mice, and a graph (bottom) that shows that this reduced phosphorylation of ERK1/2 in ARRY-treated mice is associated with a dose-dependent reduction in epithelial and mesenchymal proliferation in lung sections.

FIG. 3 is a graph illustrating that ARRY prevents TGF-α-mediated alterations in lung mechanics, including airway resistance, airway elastance, tissue elastance, and compliance.

FIG. 4 illustrates that ARRY prevents TGF-α-induced pulmonary fibrosis as demonstrated by lung histology.

FIG. 5 illustrates that ARRY prevents TGF-α-induced fibrosis as assessed by total lung collagen.

FIG. 6 is a graph showing that body weight loss was attenuated in mice having TGF-α-induced pulmonary fibrosis after being treated with ARRY, compared with vehicle-treated mice.

FIG. 7 is a series of graphs illustrating that mice having TGF-α-induced pulmonary fibrosis after being treated with ARRY demonstrated improved lung mechanics (compliance, airway resistance, elastance) compared with vehicle-treated mice.

FIG. 8 shows that mice having TGF-α-induced pulmonary fibrosis have reduced pulmonary fibrosis after being treated with ARRY, as measured by lung matrix expression on lung histology.

### DETAILED DESCRIPTION OF THE INVENTION

### Definitions

As used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

“Administering” when used in conjunction with a therapeutic means to administer a therapeutic systemically or locally, as directly into or onto a target tissue, or to administer a therapeutic as a compound or composition containing the therapeutic to a patient whereby the therapeutic positively impacts the tissue to which it is targeted. A compound or composition containing the therapeutic may be administered by injection, topical administration, and oral administration or by other methods alone or in combination with other known techniques.

The terms “ameliorate” or “ameliorating” mean to make or become better, or to improve.

The term “animal” as used herein includes, but is not limited to, humans and non-human vertebrates such as wild, domestic and farm animals.

The term “inhibiting” includes the administration of a therapeutic as a compound or composition containing the therapeutic of the present disclosure to prevent the onset of symptoms, alleviate symptoms, or eliminate the disease, condition or disorder.

“Optional” or “optionally” may be taken to mean that the subsequently described structure, event or circumstance may or may not occur, and that the description includes instances where the events occurs and instances where it does not.

The terms “patient” and “subject” are interchangeable and may be taken to mean any living organism which may be treated with compounds of the present disclosure. As such, the terms “patient” and “subject” may include, but are not limited to, any non-human mammal, any primate or a human.

By “pharmaceutically acceptable”, it is meant the carrier, diluent or excipient must be compatible with the other ingredients of the formulation and not deleterious to the recipient thereof.

The term “pharmaceutical composition” means a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “prevent” means to stop or hinder something from happening, especially by advance planning or action, or prophylactic treatment. Prevention implies anticipatory counteraction, such as by administering a therapeutic or a composition containing a therapeutic to a patient at risk of developing a disease that is preventable via the therapeutic.

The term “progressing” means developing into a more advanced stage. For example, in referring to “progressing pulmonary fibrosis” in a patient, the fibrosis exemplified is more advanced, or shows worsening disease, as compared to that of a patient with little or no pulmonary fibrosis.

The term “pronounced” means at a noticeably and markedly advanced stage. For example, in referring to “pronounced pulmonary fibrosis” in a patient, the fibrosis exemplified is markedly advanced, or shows advanced disease, as compared to that of a patient with little or no pulmonary fibrosis.

As used herein, the term “therapeutic” means an agent, compound, or composition containing the therapeutic utilized to treat, combat, ameliorate, prevent or improve an unwanted condition or disease of a patient. In part, embodiments of the present invention are directed to the treatment of pulmonary fibrosis and/or the amelioration of the symptoms of pulmonary fibrosis.

A “therapeutically effective amount” of compound of this invention is typically an amount such that, when administered in a physiologically tolerable excipient composition, is sufficient to achieve an effective systemic concentration or local concentration in the tissue and elicit a biological or medicinal response in the tissue, system, animal, individual or human to which it was administered.

The terms “treat”, “treated”, “treatment” or “treating” as used herein refers to both therapeutic treatment and prophylactic or preventative measures, wherein the object is to prevent or slow (lessen) an undesired physiological condition, disorder or disease without excessive levels of side effects, or to obtain beneficial or desired clinical results. The terms also include prolonging survival, as compared to expected survival if not receiving treatment.

For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease.

The activity contemplated by the present methods includes both medical therapeutic and/or prophylactic treatment, as appropriate. The compounds are effective over a wide dosage range and, for example, dosages per day will normally fall within the range of from 0.001 to 1000 mg/kg, more usually in the range of from 0.01 to 30 mg/kg. In some instances dosage levels below the lower limit of the aforesaid ranges may be more than adequate, while in other cases still larger doses may be employed without causing any harmful side effects, and therefore the above dosage ranges are not intended to limit the scope of the invention in any way. Subjects can receive 1, 2, 3 or more doses daily, for up to a week, 2 weeks, 3 weeks, 4 weeks, or longer. It will be understood that the amount of the compound actually administered will be determined by a physician, in the light of the relevant circumstances, including the condition to be treated, the chosen route of administration, the actual compound or compounds administered, the age, weight, and response of the individual patient, and the severity of the patient's symptoms.

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

### DETAILED DESCRIPTION OF THE INVENTION

The “mitogen-activated protein kinase”/“extracellular signal-regulated kinase” (MAPK/ERK, or just MAPK) pathways are signal transduction pathways that couple intracellular responses to the binding of growth factors (such as EGF) to cell surface receptors (such as EGFR). The MAPK pathways are one of the major downstream pathways controlling cellular processes associated with fibrosis, including cell growth, proliferation, differentiation, migration, protection from apoptosis, and transformation. There are several distinct MAPK pathways, important in the regulation of cell proliferation, differentiation, development, inflammation, survival, and migration.

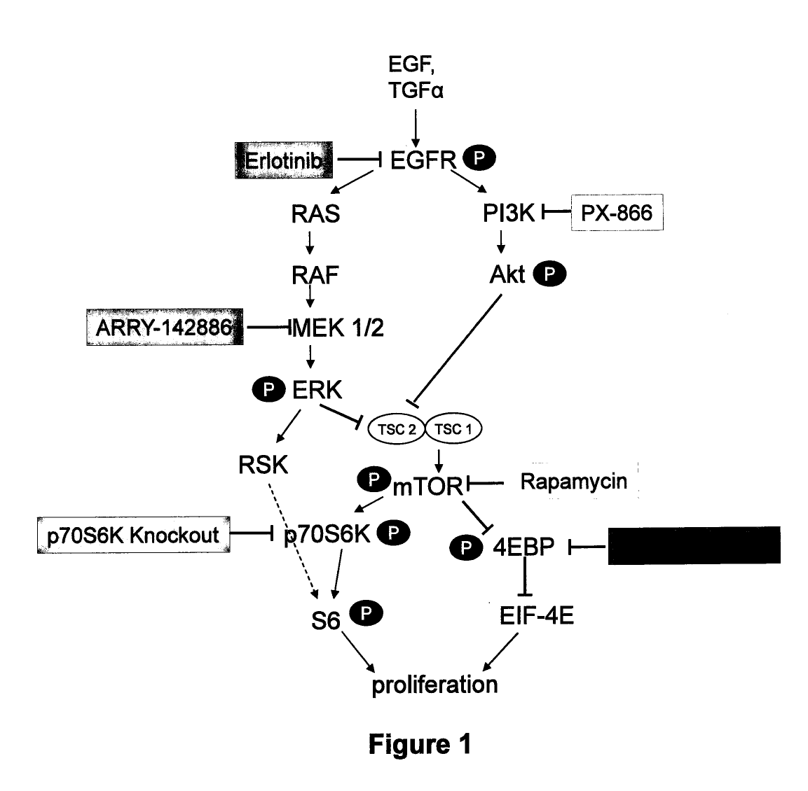

FIG. 1 is a diagram showing key components of the MAPK/ERK signaling pathways, and inhibitors of those components. In the diagram, “P” represents phosphate. In transformed cells, the Ras-Raf-MEK-ERK pathway has been implicated in cell proliferation and survival. The Ras-Raf-MEK-ERK pathway is activated by a range of growth factor receptors (including EGFR, platelet-derived growth factor receptor, type-1 insulin-like growth factor receptor, and fibroblast growth factor receptor). The pathway can also be activated by cytokines, steroid hormones, and several agonists that act via G-protein-coupled receptors.

As illustrated in FIG. 1, growth-factor stimulation of the MAPK pathways, such as by EGF or TGF-α, leads to sequential activation of Ras and Raf, which in turn activate MAPK kinases 1 and 2 (MEK1 and MEK2, or together, MEK1/2). MEK1/2 is a dual-specificity kinase that phosphorylates mitogen-activated protein kinases (MAPK's) and extracellular signal-related kinases (ERK's). MEK1/2 is essential to the propagation of growth factor signaling and is known to amplify signals to extracellular signal-regulated kinases 1 and 2 (ERK1/2, also known as MAPK1/2). MAPK's are part of a major signal transduction route that, upon activation, can phosphorylate a variety of intracellular targets including transcription factors, transcriptional adaptor proteins, membrane and cytoplasmic substrates, and other protein kinases. MAPK's transfer and amplify messages from the cell surface to the nucleus, producing a range of cellular effects, including cell proliferation.

According to the present invention, and without being limited to a specific theory, it is believed that inhibition of the Ras-Raf-MEK-ERK pathway via inhibition of MEK1/2 is an attractive strategy for therapeutic intervention in idiopathic pulmonary fibrosis. This is because inhibition of MEK1/2 has the potential to block inappropriate signal transduction leading to pulmonary fibrosis, regardless of the upstream position of the aberration causing it. Furthermore, extracellular signal-regulated kinases 1 and 2 (ERK1/2) are the only known substrates for MEK1/2.

Previously, doxycycline (Dox) regulatable transgenic mice have been generated, wherein lung epithelial-specific expression of TGF-α caused progressive and extensive vascular adventitial, peribronchial, interstitial and pleural fibrosis that was independent of inflammatory or developmental influences. Gene expression profiles observed after expression of TGF-α in the mouse lung were similar to those found in pulmonary fibrotic disease in humans. Further, it has been shown that extracellular signal-related ERK1/2 remain phosphorylated in TGF-α mice treated with PX-866 and rapamycin, supporting the theory that the MAPK/ERK pathway is a candidate as an alternative fibrosis pathway, independent of PI3K/mTOR (See FIG. 1).

For the present invention it was first determined if the MAPK/ERK pathway is activated by expression of TGF-α in the lungs of transgenic mice. The role of MEK1/2MEK1/2 in the initiation and propagation of pulmonary fibrosis was then determined by administering ARRY-142886 at the time of TGF-α induction. Finally, the effectiveness of ARRY-142886 was evaluated as a late treatment for established and progressive fibrosis in the TGF-α model.

To isolate the role of MAPK in TGF-α-mediated fibrosis, the present invention involves administering the MEK1/2MEK1/2 kinase inhibitor ARRY142886 (“ARRY” herein; also known as AZD6244), a potent, highly specific, allosteric inhibitor of MEK1/2. ARRY is a second generation MEK1/2MEK1/2 kinase inhibitor that targets the Ras-Raf-MEK-ERK pathway. This compound is known to be an orally active, potent, selective MEK inhibitor that blocks signal transductions pathways implicated in cancer cell proliferation and survival. This compound is disclosed as an MEK inhibitor for treating cancer in U.S. Patent Publication No. 2009/0099174, and can be prepared according to the process disclosed in International Patent Publication Number WO03/077914, the disclosures of which are incorporated herein by reference in their entirety.

Increased expression of EGFR ligands and activation of EGFR have been implicated in the pathogenesis of pulmonary fibrosis in a number of animal models, including bleomycin-, naphthalene-, asbestosis- and ovalbumin-induced lung injury. Signaling pathways downstream of EGFR have not been identified in these models. Our findings demonstrate the activation of the MAPK/ERK pathway in association with the induction of EGFR-mediated pulmonary fibrosis. The efficacy of ARRY142886 in preventing TGF-α-induced pulmonary fibrosis was similar to a previous study where the EGFR inhibitors gefitinib and erlotinib (See FIG. 1) also prevented fibrosis in the CCSP/TGF-α transgenic model. Together, these data support MEK1/2 as a major effector of EGFR-mediated pulmonary fibrosis.

In pulmonary fibrosis models there is limited data on activation of MEK1/2 and the effectiveness of ARRY treatment. CCSP/TGF-α transgenic mice develop fibrosis independent of inflammation. Therefore, the efficacy of ARRY in this study is unlikely to be attributed to the anti-inflammatory properties of ARRY. Fibrosis in the CCSP/TGF-α transgenic model is progressive allowing assessment of ARRY in reversing established and accumulating fibrosis.

The MAPK/ERK pathway regulates the rate of cell growth, proliferation and protein synthesis in a number of cell lines including fibroblasts, vascular smooth muscle and epithelial cells. Pulmonary fibrosis in the TGF-α model is characterized by epithelial and mesenchymal proliferation and increased extracellular matrix deposition, and both processes were effectively inhibited with ARRY administration in the prevention studies. Additional studies were then done to determine the effectiveness of MEK1/2 inhibition in established and ongoing lung fibrosis using a specific pathway component inhibitor such as ARRY.

A 4-week study was performed to evaluate the physiologic effects of MEK1/2 kinase inhibition with ARRY on TGF-α-induced pulmonary fibrosis. ARRY was administered to TGF-α transgenic mice; a model for studying pulmonary fibrosis. Endpoints included changes in lung mechanics, lung histology, lung cell proliferation and changes in matrix gene expression (Collagens I, III and Elastin). Differences in the parameters were compared between mice treated with and without ARRY. Mice which over express TGF-α that were treated with 4 weeks of vehicle (i.e. an innocuous or inert medication) developed severe lung fibrosis on histology and lung collagen measurements associated with severe lung restriction on lung mechanics. Mice treated with 4 weeks of twice daily ARRY142886 demonstrated complete prevention of lung fibrosis, including no changes in lung mechanics, and demonstrated significantly reduced lung proliferation and matrix gene expression compared to mice treated with vehicle. Together, these findings support that MEK1/2 inhibition prevents TGF-α/EGF-mediated pulmonary fibrosis, and is a possible pharmacological strategy to treat pulmonary fibrosis.

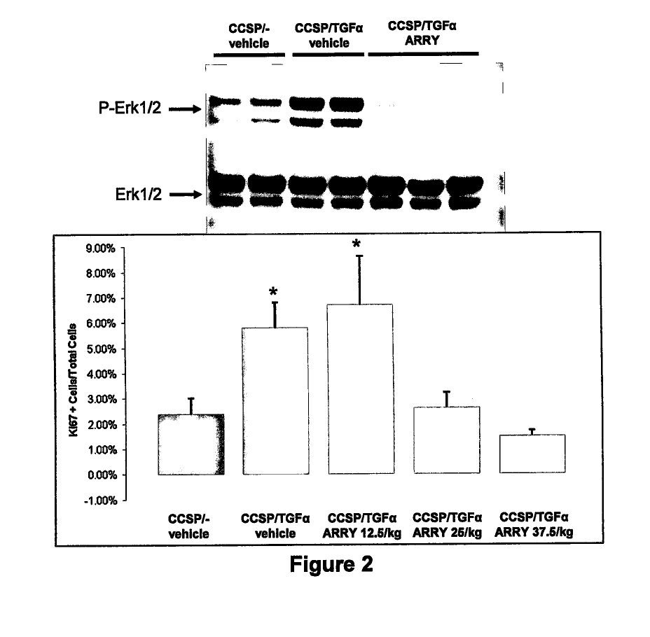

ARRY was administered to CCSP/TGF-α mice (i.e., mice expressing TGF-α in the epithelium, under control of the doxycycline (Dox)-regulatable Clara cell secretory protein promoter) twice daily by gavage, with the first dose at the time of TGF-α induction with doxycycline. Mice were sacrificed after 4 days of TGF-α induction and ARRY administration. ARRY inhibited increased phosphorylation of ERK1/2 in lung homogenates following induction of TGF-α, and caused a dose-dependent inhibition of TGF-α-induced cellular proliferation See FIG. 2, illustrating a Western blot of lung homogenates (top) showing that CCSP/TGF-α mice administered the MEK1/2 kinase inhibitor ARRY142886 (“ARRY”) demonstrate reduced phosphorylation of ERK1/2 on Western blot of lung homogenates (top) compared with vehicle-treated CCSP/TGF-α mice, and a graph (bottom) that shows that this reduced phosphorylation of ERK1/2 in ARRY-treated mice is associated with a dose-dependent reduction in epithelial and mesenchymal proliferation in lung sections (*=p<0.05 compared with CCSP/− vehicle).

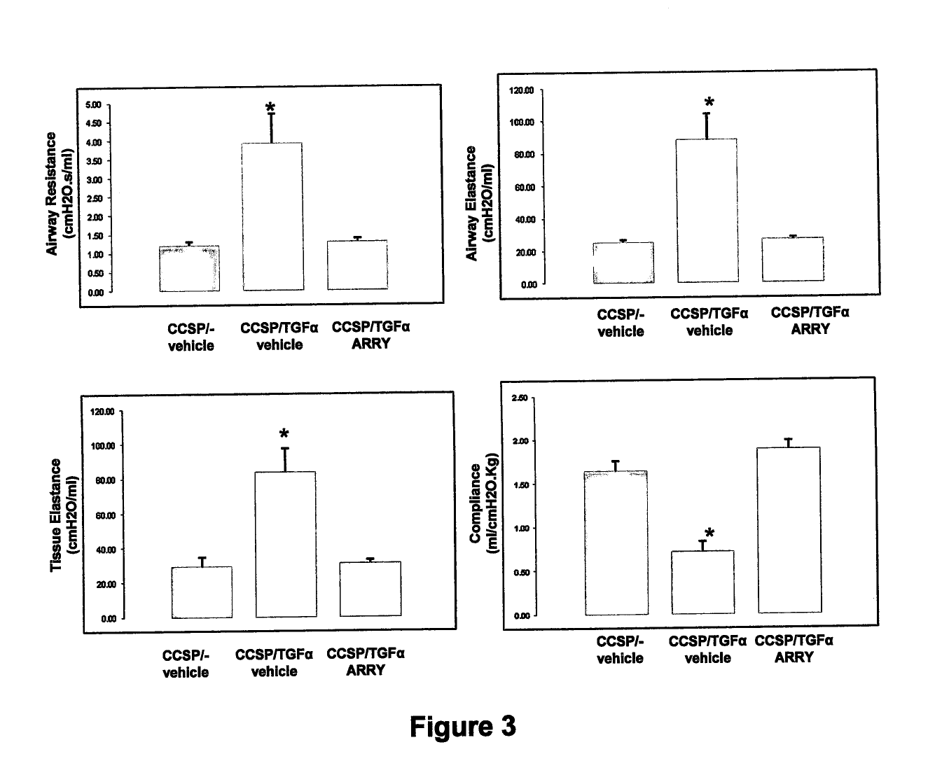

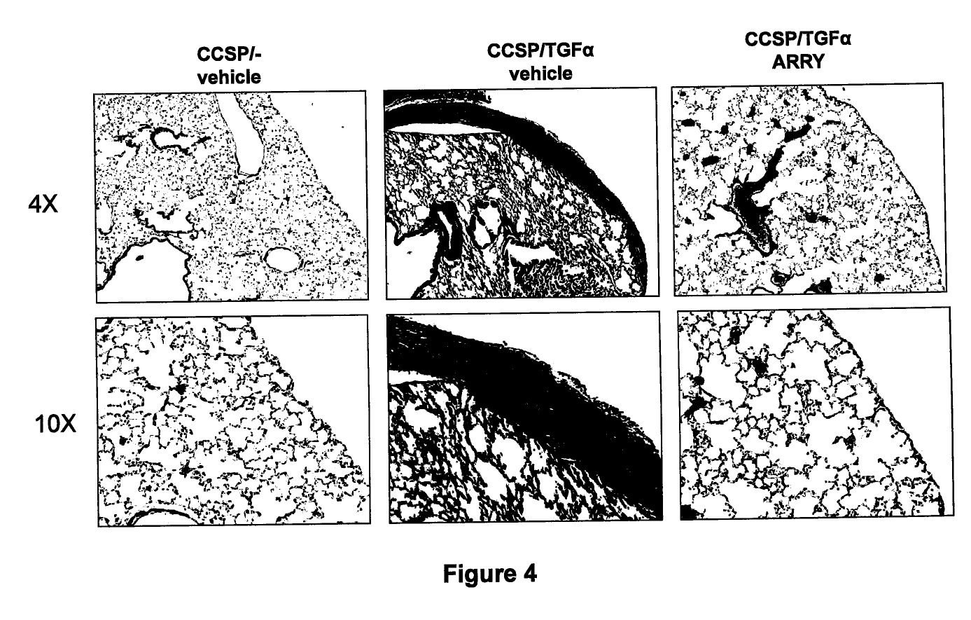

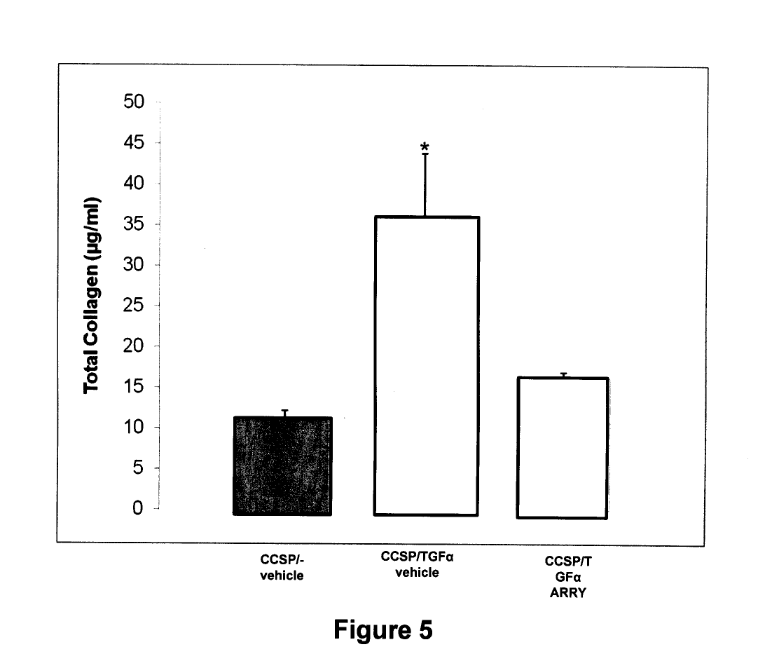

To examine if ARRY prevented the physiologic effects of TGF-α-induced lung fibrosis, CCSP/TGF-α mice were given ARRY twice daily for 4 weeks while TGF-α was induced. ARRY-treated mice had significantly reduced lung fibrosis as assessed by lung mechanics (see FIG. 3), lung histology (FIG. 4) and total lung collagen (FIG. 5). More specifically, FIG. 3 illustrates that ARRY prevents TGF-α-mediated alterations in lung mechanics (*=p<0.05 compared to vehicle and ARRY-treated mice). ARRY administered twice daily to CCSP/TGF-α mice at the time of Dox treatment prevented TGFα-mediated increases in airway resistance, and airway and tissue elastance and decreases in compliance compared with CCSP/TGF-α receiving vehicle. FIG. 4 illustrates that ARRY prevents TGF-α-induced pulmonary fibrosis as demonstrated by lung histology using a trichrome stain. Representative samples from four mice per group include low power (4×) and higher power (10×) of pleural fibrosis. FIG. 5 illustrates that ARRY prevents TGF-α-induced fibrosis as assessed by total lung collagen (*=p<0.05 compared to vehicle and ARRY-treated mice).

Together these data demonstrate that ARRY142886 can selectively inhibit the MAPK/ERK pathway in vivo and prevent TGF-α-mediated fibrosis. As the MAPK/ERK pathway is also induced by other fibrogenic growth factors, such as transforming growth factor beta (TGF-β) and platelet derived growth factors, these data support MAPK/ERK pathway inhibition as an attractive target to prevent or reverse fibrosis caused by a number of heterogeneous upstream fibrogenic factors.

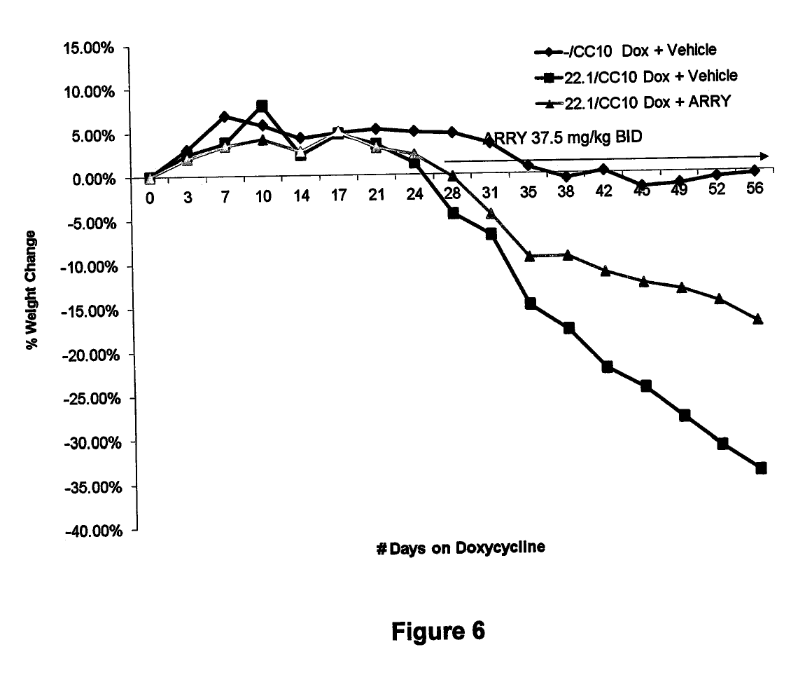

To determine whether the ERK/MAPK pathway mediates the progression of established fibrosis, CCSP/TGFα mice were treated with ARRY following 4 weeks of Dox, while remaining on Dox for an additional 4 weeks (8 weeks total Dox, 4 week ARRY). Controls included CCSP/TGFα mice treated with vehicle while remaining on Dox an additional 4 weeks (8 weeks total Dox, 4 week vehicle). CCSP/TGFα mice began losing body weight after 4 weeks of Dox. Body weight loss was attenuated in mice treated with ARRY, compared with vehicle-treated mice. See FIG. 6, illustrating that TGF-α was induced with doxycycline for 8 weeks in transgenic mice. ARRY added after 4 weeks of TGF-α expression attenuated weight loss, compared to vehicle-treated mice.

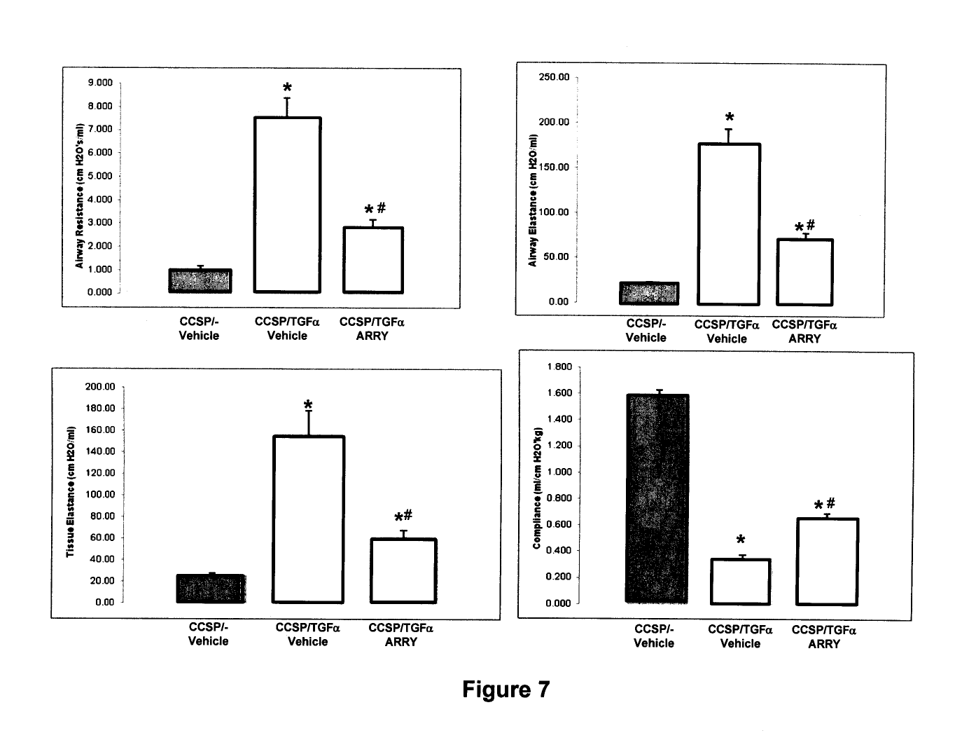

Mice treated with ARRY demonstrated improved lung mechanics (compliance, airway resistance, elastance) compared with vehicle-treated mice. See FIG. 7, illustrating that TGF-α was induced with doxycycline for 8 weeks in CCSP/TGF-α transgenic mice. ARRY added after 4 weeks of TGF-α expression prevented progression of alterations in lung mechanics compared to vehicle-treated mice. *=p<0.05 compared to vehicle control CSP/− mice. *#=p<0.05 compared to CCSP/TGF-α mice.

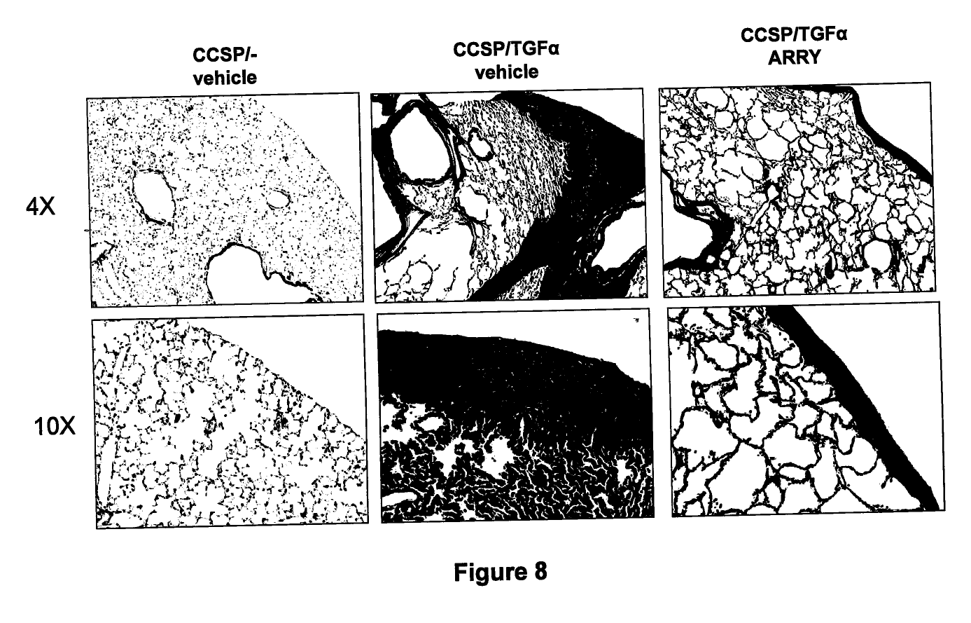

Mice treated with ARRY also demonstrated reduced pulmonary fibrosis measured by lung matrix expression on lung histology. See FIG. 8, illustrating that ARRY prevents progression of TGF-α induced fibrosis as assessed by lung histology using a trichrome stain. In FIG. 8, a representative sample from 4 mice per group includes low power (4×) and higher power (10×) of pleural fibrosis. Together these findings demonstrate that ARRY administered as a rescue treatment to mice with established and progressing fibrosis was effective in preventing the progression of fibrotic lung disease.

### Methods:

Transgenic Mice: CCSP-rtTA activator mice expressing the reverse tetracycline responsive transactivator (rtTA) under control of the 2.3-kb rat Clara Cell Secretory Protein (CCSP), a.k.a. secretoglobin, family 1A, member 1 (Scgb1a1)] gene promoter were mated to conditional Dox-regulated transgenic mice containing the human TGF-α cDNA under the control of seven copies of the tetracycline operon ((TetO)7-cmv TGF-α) plus a minimal CMV promoter. Single transgenic (CCSP-rtTA+/−) and bitransgenic CCSP-rtTA+/−/(TetO)7-cmv TGF-α+/− mice. All mice were derived from the FVB/NJ inbred strain. Mice were maintained in virus-free containment and protocols were approved by the Institutional Animal Use and Care Committee of the Cincinnati Children's Hospital Research Foundation. Mice were genotyped as is known in the art.

To induce TGF-α expression, adult transgenic mice (8-12 weeks old) were placed on Doxycycline-containing drinking water (0.5 mg/ml) and food (62.5 mg/kg). Dox-containing water was replaced three times per week.

Administration of ARRY142886: ARRY142886 (ARRY) is a potent, highly specific, allosteric inhibitor of MEK1/2. ARRY is a second generation MEK inhibitor that targets the Ras-Raf-MEK-ERK pathway. ARRY was administered to mice twice daily by gavage, with the first dose at the time of TGF-α induction with doxycycline. Mice were then anesthetized (Isoflurane; Abbott Labs, Chicago, Ill.), and 37.5 mg/kg sterile ARRY142886 was administered by gavage using a 20 gauge feeding catheter (Harvard Apparatus, Holliston, Mass.). Mice were treated with vehicle or ARRY twice daily for up to 4 weeks. Mice were weighed at the beginning of the study and at weekly intervals.

Western Blots: Western blot analysis was performed on lung homogenates of CCSP/TGF-α mice treated with 4 days of Dox. See FIG. 2. Controls were littermate single transgene CCSP/− mice treated with 1 day of Dox. Protein concentrations were assessed using a Bradford Assay and protein loaded on a 4%-20% Tris/glycine SDSPAGE (Invitrogen) and electroblotted to PVDF membranes (0.45 um: Bio-Rad). Blots were blocked with 5% nonfat dry milk in TBST (10 mM Tris, pH 8, 150 mM NaCl, 0.1% Tween 20) and incubated with antibodies against total and phosphorylated Erk (Cell Signaling Technology) and proliferating cell nuclear antigen (PCNA) (Cell Signaling Technology). Blots were washed in TBST and incubated with goat anti-rabbit horse radish peroxidase conjugated (Calbiochem, EMD Biosciences) secondary antibodies and developed on film by chemiluminescence using the ECL Plus system (Amersham Biosciences). Densitometry was performed using the volume integration function on a Phosphorlmager software Imagequant 5.2 (Molecular Dynamics, Sunnyvale, Calif.) after scanning the films.

Lung Histology and Immunohistochemistry: Mice were killed with pentobarbital sodium (65 mg/ml) euthanasia solution (Fort Dodge Animal Health, Fort Dodge, Iowa), and lungs were inflation fixed using 4% paraformaldehyde at 25 cm H2O of pressure, and then allowed to fix overnight at 4° C. Fixed lungs were then washed with phosphate-buffered saline (PBS), dehydrated through a graded series of ethanols, and processed for paraffin embedding. Sections (5 um) were loaded onto polysine slides for immunostaining, hematoxylin and eosin (H&E) staining.

Total Lung Collagen: Total lung collagen was determined by measuring total soluble collagen (Sircol Collagen Assay, Biocolor, Ireland). The left lung was homogenized in 5 ml 0.5 M acetic acid containing pepsin (1 mg/10 mg tissue; Sigma-Aldrich) and incubated (24 h; 24° C.; with 240 rpm shaking). Sircol dye was added (1 ml/100 ul; 30 min), the sample centrifuged (12,000 rpm for 12 min), and the pellet was suspended (1 ml 0.5 M NaOH). The optical density measured with a spectrophotometer (540 nm).

Pulmonary Mechanics: Lung mechanics were assessed on anesthetized mice using a computerized Flexi Vent system (SCIREQ, Montreal, Canada). Briefly, mice were anesthetized with ketamine and xylazine, tracheostomized, and then ventilated with a tidal volume of 8 ml/kg at a rate of 450 breaths/min and positive end-expiratory pressure (PEEP) of 2 cm H2O computerized by the SCIREQ system thereby permitting analysis of dynamic lung compliance. The ventilation mode was changed to forced oscillatory signal (0.5-19.6 Hz), and respiratory impedance was measured. Tissue elastance was obtained for mice at 2 cmH2O PEEP by fitting a model to each impedance spectrum. With this system, the calibration procedure removed the impedance of the equipment and tracheal tube.

Statistics: Using normal plots and tests for normality (Shapiro-Wilk and Kolmogorov-Smirnov), all response variables showed a significant departure from the normality assumption. Therefore, log transformations of the above response variables were used in order to compare group means in a one-way ANOVA. Differences in group means were calculated and tested using a simulation-based adjustment for multiple comparisons.

While the present invention has been illustrated by the description of embodiments thereof, and while the embodiments have been described in considerable detail, it is not intended to restrict or in any way limit the scope of the appended claims to such detail. Additional advantages and modifications will be readily apparent to those skilled in the art. The invention in its broader aspects is therefore not limited to the specific details, representative system and method, and illustrated examples shown and described. Accordingly, departures may be made from such details without departing from the scope or spirit of the invention.

What is claimed is:

1. A method of ameliorating symptoms associated with pulmonary fibrosis in a subject diagnosed with pulmonary fibrosis, comprising the step of administering to the subject a therapeutically effective amount of a MEK1/2 kinase inhibitor, thereby ameliorating the symptoms associated with pulmonary fibrosis in the subject.

2. The method of claim 1, wherein said MEK1/2 kinase inhibitor is ARRY142886.

3. The method of claim 1, wherein the step of administering comprises administering a composition comprising a therapeutically effective amount of the MEK1/2 kinase inhibitor.

4. The method of claim 1, wherein said MEK1/2 kinase inhibitor prevents an increase in total lung collagen.

5. The method of claim 1, wherein said MEK1/2 kinase inhibitor prevents TGF-α dependent changes in lung mechanics, said lung mechanics being selected from the group consisting of airway resistance, airway elastance, tissue elastance, and compliance.

6. The method of claim 1, wherein the type of pulmonary fibrosis is idiopathic pulmonary fibrosis.

7. The method of claim 1, wherein said MEK1/2 kinase inhibitor prevents progression of pulmonary fibrosis.

8. A method of preventing the progression of established pulmonary fibrosis in a subject diagnosed with pulmonary fibrosis, comprising the step of administering to the subject a therapeutically effective amount of a MEK1/2 kinase inhibitor, thereby preventing the progression of pulmonary fibrosis in the subject.

9. The method of claim 8, wherein the step of administering comprises administering a composition comprising a therapeutically effective amount of the MEK1/2 kinase inhibitor.

10. The method of claim 8, wherein said MEK1/2 kinase inhibitor is ARRY142886.

11. The method of claim 8, wherein said pulmonary fibrosis is either progressing or pronounced.

12. The method of claim 8, wherein said MEK1/2 kinase inhibitor slows progression of TGF-α dependent changes in lung mechanics, said lung mechanics being selected from the group consisting of airway resistance, airway elastance, tissue elastance, and compliance.

13. The method of claim 8, wherein the type of pulmonary fibrosis is idiopathic pulmonary fibrosis.

14. The method of claim 8, wherein said MEK1/2 kinase inhibitor attenuates body weight loss.

15. A method of reversing established pulmonary fibrosis in a subject diagnosed with pulmonary fibrosis, comprising the step of administering to the subject a therapeutically effective amount of a composition comprising a MEK1/2 kinase inhibitor, thereby reversing pulmonary fibrosis in the subject.

16. The method of claim 15, wherein the step of administering comprises administering as a composition comprising the MEK1/2 kinase inhibitor.

17. The method of claim 15, wherein said MEK1/2 kinase inhibitor slows progression of TGF-α dependent changes in lung mechanics, said lung mechanics being selected from the group consisting of airway resistance, airway elastance, tissue elastance, and compliance.

18. The method of claim 15, wherein said MEK1/2 kinase inhibitor prevents an increase in total lung collagen.

19. The method of claim 15, wherein said MEK1/2 kinase inhibitor prevents TGF-α dependent changes in lung mechanics, said lung mechanics being selected from the group consisting of airway resistance, airway elastance, tissue elastance, and compliance.

20. The method of claim 15, wherein said MEK1/2 kinase inhibitor is ARRY142886.
